(19) United States
(12) Patent Application Publication
(10) Pub. No.: US 2010/0222708 A1
(43) Pub. Date: Sep. 2, 2010

(54) INTRA-VAGINAL SENSOR TO MEASURE PELVIC FLOOR LOADING

(76) Inventors: Robert Hitchcock, Sandy, UT (US); Paul Johnson, Pittsburgh, PA (US); Ingrid Nygaard, Salt Lake City, UT (US); Evan Rosenbluth, Iowa City, UT (US)

Correspondence Address: Ballard Spahr LLP, SUITE 1000, 999 PEACHTREE STREET, ATLANTA, GA, 30309-3915, US

(21) Appl. No.: 12/643,438

(22) Filed: Dec. 21, 2009

Related U.S. Application Data
(60) Provisional application No. 61/139,382, filed on Dec. 19, 2008.

(51) Int. Cl. A61B 5/00

(52) U.S. Cl. 600/591; 600/587

(57) ABSTRACT

Devices and methods are provided for measuring pressure in a non-fluid-filled area of a patient's body. The device includes a capsule having an outer wall and a base surface that define an inner cavity, which retains fluid. The outer wall of the capsule can be made of a flexible material. The device also has a pressure sensor that measures external pressure placed on the outer wall of the capsule. Methods of use include providing the device, positioning the device in the non-fluid-filled area, and measuring pressure within the non-fluid-filled area. The pressure measurement is compared with a predetermined pressure threshold, and a treatment regimen is generated based on the differences between the measured pressure and the predetermined pressure threshold.

### CROSS REFERENCE TO RELATED APPLICATIONS

This application claims priority to and the benefit of U.S. Provisional Application No. 61/139,382, filed on Dec. 19, 2008, which is incorporated in its entirety in this document by reference.

### FIELD OF THE INVENTION

This invention relates generally to devices and methods for measuring pressures therein areas of a patient's body. More specifically, devices and methods are provided for measuring intra-abdominal pressure of a patient.

### BACKGROUND

At the base of the abdominal compartment lies a group of muscles commonly referred to as the pelvic floor muscles. These muscles serve as a hammock-like structure, supporting the intra-abdominal matter. Pelvic floor disorders occur when these muscles become weakened or damaged, thereby affecting the positioning and function of the supported organs. In recent years, pelvic floor disorders among women have become increasingly prevalent.

In an attempt to minimize incidence and recurrence of pelvic floor disorders, doctors commonly prescribe physical activity restrictions to those at risk based on a supposition that certain physical activities significantly raise intra-abdominal pressure and place excessive strain on the pelvic floor muscles. Despite general certainty that physical activity can raise pressure in the abdomen, there is much uncertainty as to which activities promote pelvic floor dysfunction, thereby resulting in significant variations in the activity restrictions prescribed to patients by their doctors.

Current technology does not allow for accurate measurement of the types of loads that the pelvic floor muscles support during daily life. In this context, it is appreciated that intravaginal pressures are a proxy for intra-abdominal pressures. The current technology generally requires that a patient be awkwardly attached to laboratory based diagnostic instruments during testing, thus deviating from real-world simulation. Other existing devices require nearly perfect placement within the patient in order to obtain accurate measurements, are highly invasive, or are so sensitive that natural movement by the patient during testing can affect the accuracy of the results.

Thus, there is a need in the art for minimally invasive devices and methods for accurately measuring pressure within areas of the body under real-world simulation.

### SUMMARY OF THE INVENTION

Devices and methods are provided for measuring pressure within an area of a patient. According to various aspects, the area of the patient can be a non-fluid-filled area or a fluid-filled area of the patient. In one aspect, the devices and methods are provided for measuring the intra-vaginal pressure of a patient. According to various aspects, the device comprises a capsule and a pressure sensor. The capsule can have an outer wall and a base surface, which cooperate to define an inner cavity configured to retain a fluid. At least a portion of the outer wall, in one aspect, comprises a flexible material. The pressure sensor can be configured to measure external pressure placed on the outer wall of the capsule. In one aspect, the pressure sensor can be positioned therein the inner cavity of the capsule.

Also disclosed are methods of using an exemplary device for measuring pressure within an area of a patient, such as, but not limited to, a non-fluid-filled or fluid-filled area of the patient. Exemplary methods can comprise providing a device and positioning the device within the area of the patient. The method can further comprise measuring pressure within the area and comparing the pressure measurement with a predetermined pressure threshold. A treatment regimen can be generated based on the differences between the measured pressure and the predetermined pressure threshold.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several aspects of the invention and together with the description, serve to explain the principles of the invention.

FIG. 1 illustrates a perspective view of an exemplary intra-vaginal sensor.

FIG. 2 illustrates a perspective view of the exemplary intra-vaginal sensor of FIG. 1.

FIG. 3 illustrates an exploded perspective view of the exemplary intra-vaginal sensor of FIG. 1.

FIG. 4 illustrates a cross-sectional view of the exemplary intra-vaginal sensor of FIG. 1.

FIG. 5 illustrates use of an exemplary system for measuring intra-abdominal pressures of a patient.

FIG. 6 illustrates an exemplary placement of an intra-vaginal sensor within the vagina of a subject.

FIG. 7 is a graph of sensor sensitivity at various pressure levels.

FIG. 8 is a series of graphs illustrating frequency responses of an exemplary intra-vaginal sensor, standard rectal transducer, and reference transducer during gradual pressure changes.

FIG. 9 is a graph illustrating frequency responses of an exemplary intra-vaginal sensor and a standard rectal transducer during an impulse response test.

FIG. 10 is a graph of the changes in pressure measured by an exemplary intra-vaginal sensor and a standard rectal transducer while a subject lifted ten pounds.

FIG. 11 is a graph of the changes in pressure measured by an exemplary intra-vaginal sensor and a standard rectal transducer while a subject engaged in a series of jumps.

FIG. 12 is a graph comparing the pressure measurements of an exemplary intra-vaginal sensor with the pressure measurements of a rectal balloon catheter while a subject engaged in various physical activities.

### DETAILED DESCRIPTION

The present invention may be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Thus, for example, reference to a “sensor” can include two or more such sensors unless the context indicates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

Reference will now be made in detail to the various aspects, examples of which are illustrated in the accompanying drawings. Wherever possible, the same reference numbers are used throughout the drawings to refer to the same or like parts.

As described above, devices and methods are provided for measuring the pressure therein areas of a patient's body. According to various aspects, the areas of the patient's body can be non-fluid-filled areas. The term “non-fluid-filled” is intended to mean areas of the body that are not completely filled with fluid, but which may be partially filled with fluid, or absent of fluid. The term “non-fluid-filled” can also refer to areas of the body that can be temporarily filled with fluid at specific times, but which may be partially filled with fluid or absent of fluid at other times. Exemplary non-fluid-filled areas of the patient's body can include, but are not limited to, organs such as the esophagus, large intestine, colon, vagina, uterus, as well as musculoskeletal structures and/or other areas. Thus, although described herein with reference to measuring intra-vaginal pressure, it is contemplated that the devices and methods described herein are not intended to be limited to measuring pressure in any one area of the body.

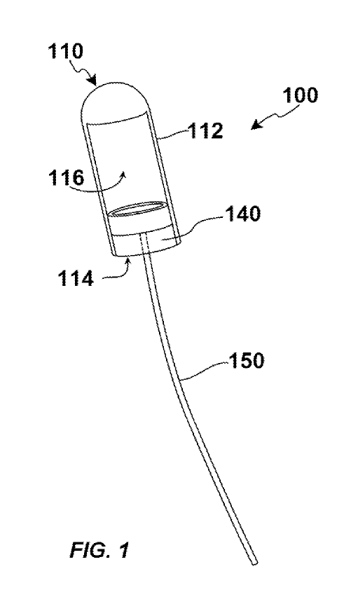

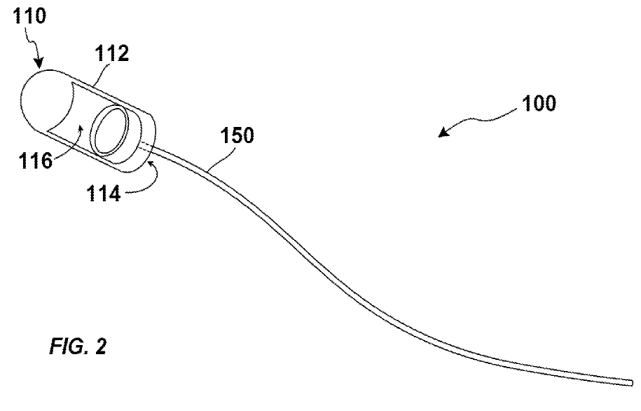

According to various aspects, a device is provided for measuring pressure therein an area of a patient's body, such as, but not limited to, a non-fluid-filled area. With reference to FIGS. 1-3, the device 100 in one aspect comprises a capsule 110. The capsule 110 can have an outer wall 112 and a base surface 114, which can cooperate to define an inner cavity 116. The inner cavity, in one aspect, is configured to retain a fluid. The fluid, in one aspect, can be silicone gel. Alternatively, and without limitation, the fluid can be one or more of the following: water, saline, silicone oil, or other similar pressure-transmitting fluids.

The device can further comprise a pressure sensor 122 that is configured to measure external pressure placed on the outer wall of the capsule. According to various aspects, the pressure sensor can be an optical pressure sensor, radio frequency (RF) sensor, or other known pressure sensor. In one exemplary aspect, the pressure sensor can be a Novasensor P562 micro-sensor. In a further aspect, the pressure sensor can comprise a fluid chamber configured to contain a fluid. The pressure sensor, in one aspect, is positioned therein the inner cavity 116 of the capsule. For example, in one aspect, the pressure sensor can be mounted on a circuit board 120, as described further herein below. According to various aspects, the capsule 110 is configured to be positioned therein the non-fluid-filled area of the patient to measure pressure therein the non-fluid-filled area.

According to another aspect, the capsule 110 can further comprise a ring 130 configured to be positioned therein the capsule proximate the base surface 114 of the capsule and provide a surface for surrounding the circuit board 120, which contains the pressure sensor 122 as well as conventional electronic components 124 such as amplifiers, resistors, capacitors, transceiver, wires and batteries. In one exemplary aspect, the ring can be comprised of aluminum. The ring, in one aspect, can be filled with a compliant gel 132 that faithfully transmits pressure applied onto the ring and the gel therein to the pressure sensor. Thus, in one aspect, the ring, circuit board, and outer wall 112 can cooperate to retain the fluid therein the inner cavity 116 of the capsule.

In one aspect, and with reference to FIG. 1, the capsule 110 comprises a proximal end portion, an opposing distal end portion, and a body portion that extends therebetween the proximal end portion and the distal end portion. In one aspect, the distal end portion can be substantially hemi-spherical and the body portion and proximal end portion can be substantially cylindrical and can extend along a longitudinal axis of the capsule. Thus, in one aspect, the capsule can be substantially bullet-shaped, having a rounded distal end portion. As can be appreciated, the hemi-spherical distal end portion can have a familiar tampon-like size and shape that promotes patient comfort. In addition, the hemi-spherical distal end portion and the cylindrical proximal end portion can allow for easier insertion and retention therein the vagina of a patient.

In one aspect, the diameter of the cylindrical body portion of the capsule 110 can be between about 0.5 centimeters and 1.5 centimeters, including 0.5 cm, 0.6 cm, 0.7 cm, 0.8 cm, 0.9 cm, 1.0 cm, 1.1 cm, 1.2 cm, 1.3 cm, 1.4 cm, and 1.5 cm. According to a particular aspect, the diameter of the cylindrical body portion can be about 1.2 cm. According to various aspects, the length of the capsule taken along the longitudinal axis can be between about 2 cm and about 3 cm, including 2.0 cm, 2.1 cm, 2.2 cm, 2.3 cm, 2.4 cm, 2.5 cm, 2.6 cm, 2.7 cm, 2.8 cm, 2.9 cm, and 3.0 cm. According to a particular aspect, the length can be about 3.0 cm. It is, of course, contemplated that the diameter and length of the capsule can vary and can be selected on a per-patient basis. Additional capsule dimensions can be contemplated that would provide appropriate dimensions for devices to be used in alternate body locations. In one example, without limitation, a bullet shaped capsule with dimensions of 3 cm diameter and 8 cm in length could be used for pressure monitoring in the lower GI tract. In another example, without limitation, a capsule with dimensions of 0.3 cm diameter and a length of 1 cm could be used for monitoring pressure within parenchymal brain tissue.

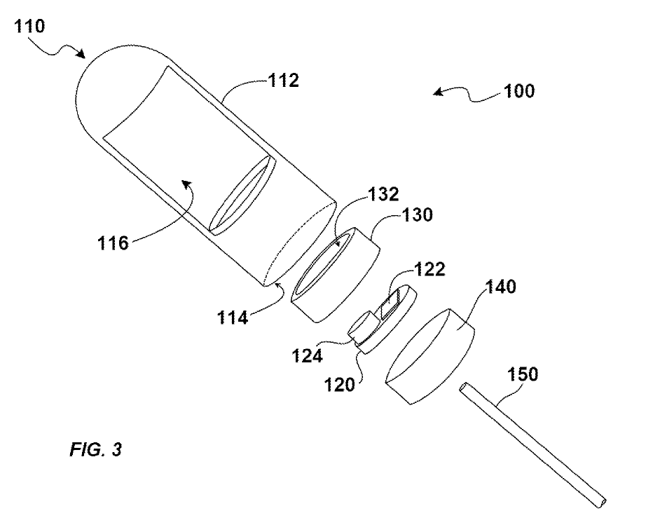

In one aspect, the pressure sensor 122 is configured to be positioned therein the fluid that is retained in the inner cavity 116 of the capsule 110. In one exemplary aspect, and not meant to be limiting, the pressure sensor can be attached to a circuit board 120, such as illustrated in FIG. 3, which is attached to the bottom of the ring 130. In this aspect, the ring is filled with a compliant, dielectric silicone gel 132 that protects the electrical circuitry and allows the transmission of pressure from the top of the gel-filled ring to the pressure sensor that is arranged at the base of the gel-filled ring. One would appreciate that, by mounting the assembly consisting of the circuit board, ring and gel into the capsule, the pressure sensor 122 is positioned within the fluid retained therein the inner cavity of the capsule. It is contemplated that the pressure sensor can be otherwise positioned within the capsule, such as, but not limited to, being suspended within the fluid of the capsule. Thus, in one aspect, and as described in further detail below, the capsule 110 can be configured to be positioned therein a non-fluid-filled area of the patient and can measure pressure therein the non-fluid-filled area.

Optionally, it is further contemplated that the entire capsule can be filled with compliant gel. As one can appreciate, the placement of the pressure sensor within a compliant, fluid-filled capsule at a physiological pressure source allows for accurate pressure measurements that are not altered by directional effects.

In one aspect, at least a portion of the outer wall 112 of the capsule 110 comprises a flexible material. For example and not meant to be limiting, the thickness of the flexible material forming the outer wall can be between about 0.3 and 1.5 mm, including 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, and 1.5 mm In a further aspect, the flexible material can comprise an elastomeric material which can be, for example without limitation, a silicone elastomer (such as a silicone elastomer having a hardness of 50 Shore A, for example, LSR-4350 Factor II).

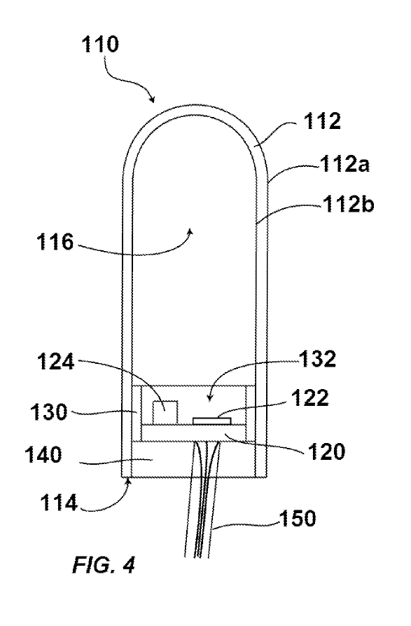

In one exemplary aspect, and with reference to FIG. 4, the outer wall 112 of the capsule can comprise an outer wall surface portion 112a and an inner wall surface portion 112b. In one aspect, the inner wall surface portion 112b of the capsule can be coated with solvent-dispersed polyvinylidene fluoride to reduce aqueous diffusion across the capsule wall. Alternatively, an ultra-thin (about 25 μm) polyethylene liner can be integrated into the inner capsule wall. According to various aspects, the capsule 110 can be configured to deform under loads typically experienced in the vagina or other targeted areas of the patient's body. As can be appreciated, the deformations of the outer wall 112 of the capsule results in increased fluid pressure therein the capsule. The increased fluid pressure can be measured by the pressure sensor 122. Thus, the pressure within the area, such as a non-fluid-filled area, of the patient's body can be measured.

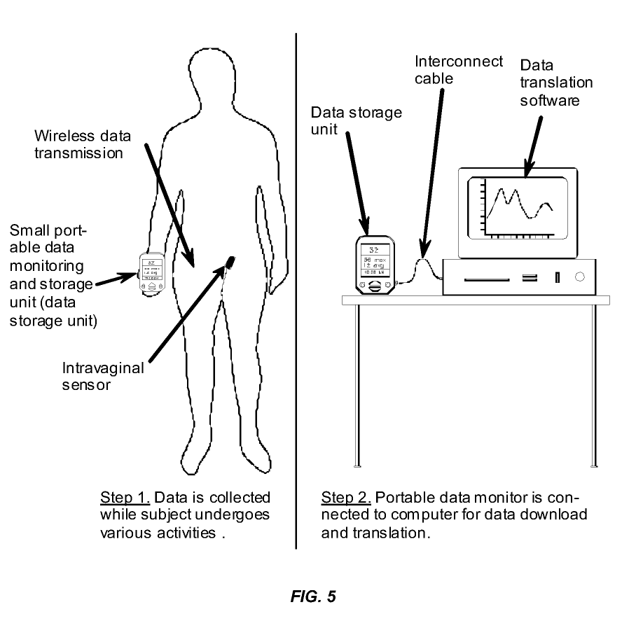

In one aspect, the sensor can be in wired communication with an external data acquisition device. Alternatively, the sensor can be configured for wireless communication with an external data acquisition device, and can be configured to transmit the sensed pressure measurement to the external data acquisition device. For example, in one aspect, a wireless system can be used in which the pressure sensor and the external data acquisition device (receiver) have internal power, provide high data bandwith, and operate in ranges of from about 0.2 m to about 10 m. However, such systems typically require batteries that must be charged or replaced, and thus necessitate larger packages or housings. Smaller implantable systems can be used that are based on passive telemetry, in which the primary node powers the secondary node via electromagnetic coupling and use backscatter amplitude modulation to detect signal changes. Such exemplary systems do not require batteries, and thus have longer lifetime and are of a smaller size. In one exemplary aspect, and not meant to be limiting, the wireless circuitry employed by the sensor and external data acquisition device can be based on the Zarlink ZL70101 MICS transceiver chip. In order to promote ease of use by consumers, an exemplary data acquisition device can comprise user and computer interface hardware. Following wireless data transmission, and as shown in FIG. 5, the external data acquisition device can be connected to a computer to download and translate the data into a spreadsheet, database, statistical software package, or technical computing environment. In one exemplary aspect, wireless transmissions of data can occur as block transfers, where, rather than relying on continuous transmissions, the sensor continuously records and stores data but periodically sends the data as a block of information to the data acquisition device.

As illustrated in FIGS. 3 and 4 and generally described above, the pressure sensor 122 can be mounted on a printed circuit board (PCB) 120. In an exemplary aspect, the base material for the circuit board 120 can be FR-4 laminate, which is a flame-retardant woven-glass-reinforced epoxy resin. As can be appreciated, the device can also comprise additional electronic components 124 as necessary, such as, but not limited to, one or more amplifiers, resistors, batteries, wireless components, or the like. In one aspect, one or more of the additional electronic components can be mounted on the PCB with the sensor. In use, the signals produced by the pressure sensor 122 can be amplified for processing, storage, and transmission.

According to another aspect, the device can further comprise a base member 140. The base member, in one aspect, can comprise a flexible, elastomeric material. In another aspect, the base member 140 can comprise a substantially cylindrical body configured to be received therein the inner cavity of the capsule proximate the base surface 114 of the capsule 110 and provide axial support to reinforce the position of circuit board 120 and ring 130 therein the capsule. In an exemplary aspect, the base member 140 can comprise a bore, such as but not limited to an axial bore, located proximate the position of the pressure sensor 122 on the circuit board.

According to yet another aspect, the device can further comprise a tether member 150. The tether member, in one aspect, can comprise a lumen having a distal end in communication with the pressure sensor 122 and an opposing proximal end in communication with an atmospheric pressure source. As can be appreciated, the tether member 150 can serve as an atmospheric reference or “vent” that allows the pressure sensor 122 to reference all readings to atmospheric pressure to generate accurate calibration and measurement. For example, the tether member in one aspect can define a lumen for providing atmospheric pressure to the pressure sensor. The tether member 150 can also comprise means for providing wired communication from the pressure sensor 122 to an external data acquisition device. In one aspect, one or more wires can extend therethrough the lumen of the tether and can be connected to the pressure sensor and/or the additional electronic components 124 at respective distal ends, and can be connected to the external data acquisition device at respective proximal ends. In this aspect, at least a portion of the tether member 150 can be positioned within the bore of the base member 140 to allow for direct communication between the pressure sensor 122 and the tether member. In an exemplary aspect, the wires can be 50 μm diameter gold-plated copper/polyurethane wires. Thus, the tether member 150 can comprise venting means and a series of wires to simultaneously allow for communication with an atmospheric pressure source and transfer of information from the pressure sensor 122 to an external data acquisition device. The tether member 150 can further comprise an integrated antenna to facilitate transmission of information to data acquisition devices.

Although a tether member can be provided to serve as a “vent” to allow the pressure sensor to reference all readings to atmospheric pressure, it is also contemplated that an absolute pressure sensor can be provided with an external pressure reference source. For example, an absolute pressure sensor can be used in a device that can be swallowed to measure pressures, such as transit pressures, in the stomach or gastrointestinal tract of a patient. The external pressure reference source can be positioned outside of the body, and can be configured to measure the absolute pressure outside of the body.

In further aspects, methods are provided for measuring pressure therein a non-fluid-filled area of a patient. An exemplary method comprises providing a device such as described herein, the device comprising a capsule 110 and a pressure sensor 122. The device can be positioned therein the non-fluid-filled area of the patient. The method can further comprise taking at least one pressure measurement based on the pressure placed on the outer wall 112 of the capsule by the non-fluid-filled area of the patient. In one aspect, the method can comprise determining if the at least one pressure measurement differs from a predetermined pressure threshold. The method can further comprise generating a treatment regimen based on the determined difference between the at least one pressure measurement and the predetermined pressure threshold.

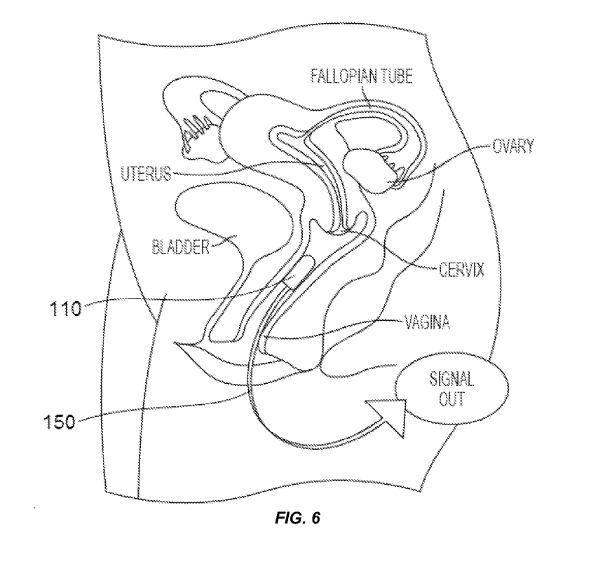

According to yet another aspect, a method is provided for measuring the intra-vaginal pressure of a patient. As depicted in FIG. 6, the method can comprise providing a device as described herein and positioning the device therein a vagina of the patient. In one aspect, the device is positioned therein the vagina above the pelvic floor of the patient. As described above, in one aspect, the device can be bullet-shaped with a rounded distal end. In this aspect, the device can be inserted therein the vagina such that the distal end portion of the capsule is proximate the cervix of the patient. In yet another aspect, the device can further comprise a tether member that is operably connected to the proximal end portion of the capsule. As can be appreciated, the tether member can be configured to assist the patient in removing the device from the patient's vagina after use. As described above, the tether member can also comprise a lumen for providing atmospheric pressure to the pressure sensor, and/or means for providing wired communication from the pressure sensor to an external data acquisition device. Optionally, a device can be provided having an absolute pressure sensor, such as described above.

The method can further comprise taking at least one pressure measurement based on the pressure placed on the outer wall of the capsule by the patient's vagina. In one aspect, the method can comprise taking at least one pressure measurement while the patient performs at least one activity. The activity can include one or more activities of the following non-exhaustive and non-limiting list: sitting, standing, walking, running, jumping, coughing, sneezing, performing a Valsalva maneuver, squatting, and/or lifting weights. The method can comprise determining if the at least one pressure measurement differs from a predetermined pressure threshold for the at least one activity. For example, for each activity, there may be a pressure or pressure range that is considered normal for a healthy patient. Thus, the determination can include determining if the at least one pressure measurement is greater than or less than the predetermined pressure threshold.

Based on the determined differences between the at least one pressure measurement and the predetermined pressure threshold for the at least one activity, a treatment regimen for the patient can be generated. For example, a patient can engage in one or more activity, and the intra-vaginal pressure can be measured while the patient engages in such activities. If the measured pressure differs from the predetermined pressure threshold for the activity, a treatment regimen can be generated advising the patient not to engage further in the activity. Optionally, the treatment regimen can advise the patient that it is safe to continue engaging in the activity.

Such methods can be used on patients following a pelvic floor surgery or as a research method to understand incidence, progression and recurrence of pelvic floor disorders. It is contemplated that a post-surgery patient can position a device in her vagina, and can engage in her usual day-to-day activities. The device can measure the pressure in the vagina while the patient engages in these activities. If the pressure measurements are determined to differ from predetermined pressure thresholds for each activity, the patient can be alerted to discontinue the activity or activities.

In one aspect, the pressure measurements can be transmitted wirelessly to an external data acquisition device, such that the patient can engage in the activities with minimal or no limitations imposed by the device itself Thus, the device can measure intra-vaginal pressure under real-world conditions, with the patient unencumbered by the device or by being tethered to external devices.

It is contemplated that devices described herein may be configured for repeated use by a single patient, and thus may be non-sterile and reusable. It is further contemplated that the devices can be washed in soap and water and reinserted during times of activity to allow for pressure measurements to be taken. Optionally, it is contemplated that the devices described herein can consist of a sterile, single-use intravaginal sensor combined with a reusable data monitor.

It is also contemplated that the devices described herein can comprise a discrete, miniature pin-accessible switch that can be used to turn the sensor on and off. It is further contemplated that the devices described herein can comprise batteries that can conserve power by automatically entering into a sleep mode during times of inactivity.

### Experimental

The following examples are put forth so as to provide those of ordinary skill in the art with a complete disclosure and description of how the devices, systems and/or methods claimed herein are made and evaluated, and are intended to be purely exemplary and are not intended to limit the scope of what the inventors regard as their invention. Efforts have been made to ensure accuracy with respect to numbers and measurements, but some errors and deviations should be accounted for.

A plurality of devices for measuring the intra-vaginal pressure of various patients were manufactured, such as described with regard to various exemplary aspects herein. The devices included capsules that were molded from silicone elastomer (50 Shore A). The capsules were bullet-shaped, having rounded proximal end portions, such as shown in FIGS. 1-3.

A piezoresistive gauge pressure sensor was selected to measure the pressure within the capsule. The sensor was mounted onto a custom-made Flame Retardant 4 (FR-4) micro printed circuit board (PCB), which provided for connections to an external power supply and an external data acquisition device. Because the FR-4 PCB exhibits thermal expansion properties different to those of the silicon sensor, one corner of the sensor was mounted to the PCB using a semi-rigid acrylic UV-cure adhesive. The remaining perimeter of the sensor was sealed to the PCB using a soft silicone UV-cure adhesive to allow for thermal expansion and contraction thereby minimizing thermal effects on the sensor output.

The PCB was then placed at the base of an aluminum ring, which was filled with platinum-based RTV silicone soft gel (available from Factor II, Inc.). A vent, providing atmospheric pressure to the gauge pressure sensor, and wires were run through a custom-made tether, which was filled with silicone elastomer and molded into the base member of the capsule. A circular connector was placed at the termination of the wires to provide connection to an extension cable and, in turn, to an Agilent 34970A Data Acquisition Switch Unit (available from Agilent Technologies, Inc.).

The testing of the prototype devices occurred during various phases (Phases 0, 1 and 2), in order to enable the design to be changed as necessary and to ensure the feasibility of the implant. Each of the phases is discussed in more detail below.

Phase 0

Phase 0 involved testing the comfort and retention of the device, the capsule load capacity, and the gel pressure transmission.

Comfort and Retention

The collapsibility of the vaginal tissue largely determines the retention of the device; however, the shape, size, and surface texture of the device can also have an effect on retention. Two devices were manufactured having a capsule formed of silicone elastomer, the capsules having a diameter of 1.2 cm and a length of 3 cm. Each capsule was placed into the vagina of a healthy female test subject at a distance of at least 5 cm into the vagina. Pressures at least 5 cm within the vagina have been verified to represent intra-abdominal pressures; the vaginal walls are subject to the same body forces exerted on the pelvic floor. Thus, it was determined that by placing the device at least 5 cm into the vagina, accurate pressure measurements of the pelvic floor can be obtained. The test subjects were instructed to participate in exercises such as running, jumping, and lifting. The test subjects were then instructed to determine if the device remained at least 5 cm within the vagina throughout exertion.

The two test subjects reported that the device was comfortable. During physical exertion, the device was confirmed to retain its position within the vagina (i.e., at least 5 cm above the vaginal opening).

Capsule Load Capacity

The desired functions of the capsule are, among others, to retain pressure, protect the intra-capsule components from the physiologic environment, to withstand the pressures of the physiologic environment and maintain its structural integrity. An optimal outer wall thickness of the capsule was sought for maximum sensitivity and load capacity. Three different capsule wall thicknesses were selected for testing: 0.64 mm (0.025 in.), 0.89 mm (0.035 in.) and 1.27 mm (0.05 in.). Three test capsules were created for each wall thickness and were filled with 2 mL of water. Using a pressure generator, each capsule was pressurized to 1680 cm H₂O (24 psi), over four times the typical intra-abdominal pressure in a healthy female. A materials test machine was then used to individually compress each capsule until failure. The material test machine load cell was able to measure up to 1000 N of force. Thus, the capsules were compressed until failure or the 1000 N limit. Results of the compression testing demonstrated that in the trials, the capsules failed under forces of over 1000 N, or did not fail.

Gel Pressure Transmission

To verify that the sensor would transmit fluid pressure measurements and maintain its sensitivity under a thin layer of gel, the sensitivity of the sensor was tested under gel layers of varying thicknesses: 2 mm, 4.1 mm and 6.3 mm. A pressure generator was utilized to deliver known pressures. Sensor response was measured using the Agilent 34970A Data Acquisition Switch Unit. The sensitivity of the sensor was reduced by less than 4% over the range of different gel thicknesses tested.

Phase 1

After Phase 0 was concluded, a fully-functioning device was tested in the laboratory. The device was pressurized in 35.1 cm H₂O (0.5 psi) increments using a pressure generator (Tescom ER 3000), and the voltage output was measured using the Agilent 34970A Data Acquisition Switch Unit in order to produce a response curve and characterize the sensitivity of the implant.

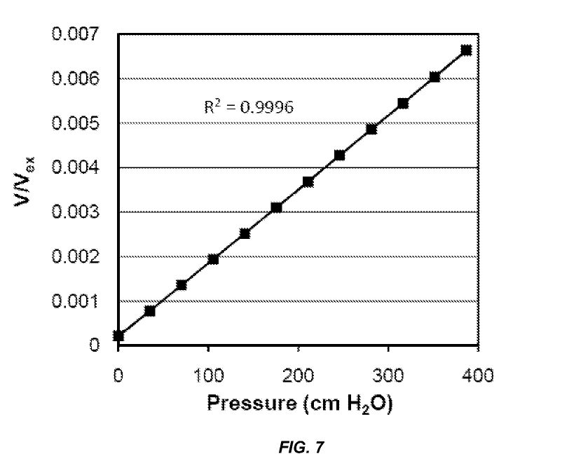

As shown in FIG. 7, the devices demonstrated a linear response from 0 to 387 cm H₂O (5.5 psi). The sensitivity of the device was determined to be approximately 2E-5 mV/V/cm H₂O.

In addition to conducting sensitivity testing, the frequency responses of a vaginal transducer and a standard rectal transducer were tested by placing both transducers in a pressure chamber. An offset pressure of 35.15 cm H2O was applied to the chamber using a pressure generator. In order to test the transducers' responses during gradual pressure changes, a sine pressure wave with an amplitude of 7 cm H2O was introduced into the chamber using a modified and sealed speaker. To test the transducers' responses during abrupt pressure changes, a square wave with the same amplitude of 7 cm H2O was introduced into the chamber. Both square and sine waves were input at clinically relevant frequencies ranging from 0.2 Hz to 3 Hz. The measurement sampling rate for the vaginal and rectal transducers was 10 Hz.

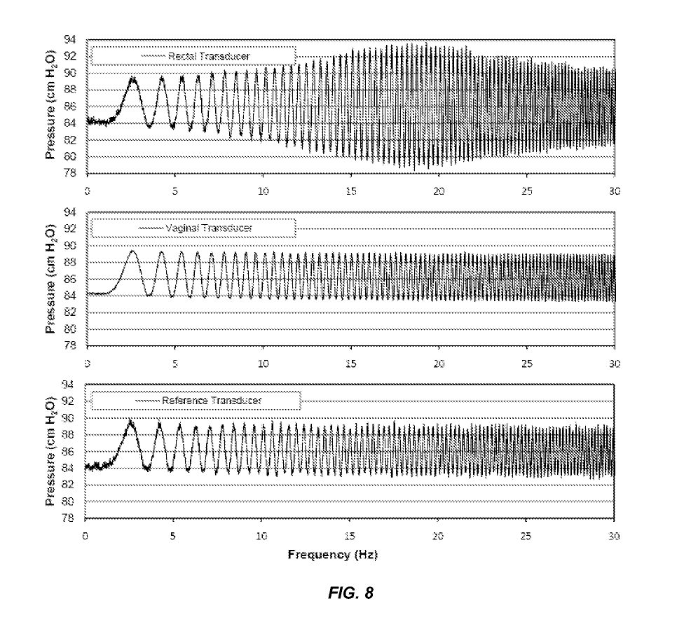

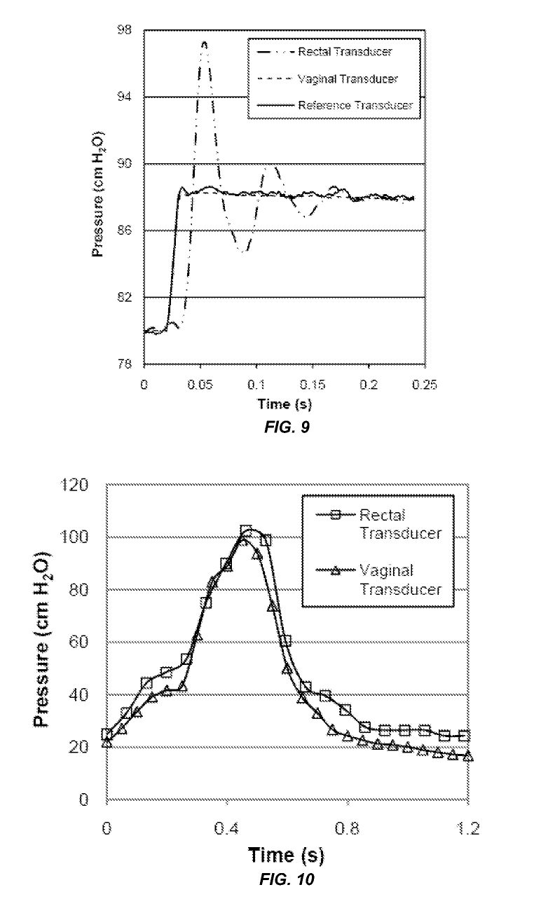

As shown in FIG. 8, during gradual pressure changes (sine waves) at lower frequencies, both the vaginal and rectal transducers accurately measured the pressure in the chamber as confirmed by a reference pressure transducer. Aliasing effects began to appear in both transducers' responses when the sine wave frequencies reached 3 Hz. Taking measurements at a higher frequency confirmed that these effects were due to the slower sampling rate used. As illustrated by FIG. 9, when abrupt pressure changes (square wave) were introduced into the pressure chamber, the vaginal transducer continued to accurately measure the pressure within the chamber while the rectal transducer began to exhibit overshoot. This agreed with previous studies reporting catheter-tubing measurement systems, which are coupled via fluid to an external transducer, to be inadequate for measuring rapidly changing pressures. The exemplary vaginal transducer described herein, however, continued to provide accurate measurements during abrupt pressure changes.

Phase 2

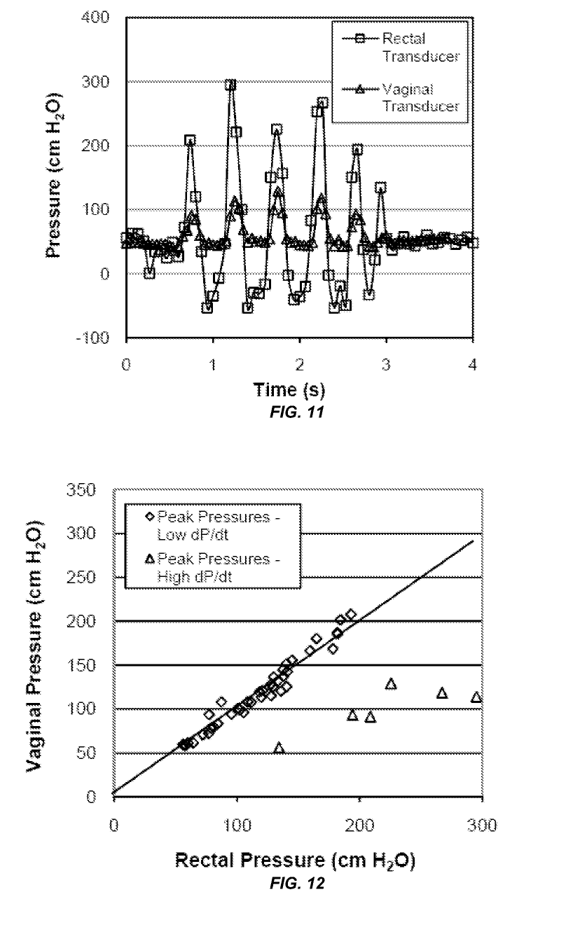

Eight fully-functioning devices were created for clinical testing. Each sensor was placed in the upper vagina of a respective test subject by practicing clinicians during urodynamics testing. During testing, a rectal catheter was also placed in the test subjects. Each of the test subjects performed routine urodynamics tasks such as, but not limited to, coughing and straining (performing a Valsalva maneuver). The test subjects were also asked to perform a series of squats, lifts, and jumps after the urodynamics tasks were performed. All tasks were performed with a bladder volume of 200 mL. FIGS. 10 and 11 display graphs of the changes in pressure measured by the intra-vaginal sensors and rectal catheters during lifts of 10 pounds, and jumping exercises, respectively.

The clinical testing of the intra-vaginal pressures that occur during various forms of physical exertion provided reliable results. As demonstrated by FIG. 12, the vaginal and rectal transducers had similar responses during physical activities inducing gradual intra-abdominal pressure changes, but the vaginal transducer had a superior response during abrupt intra-abdominal pressure changes.

The clinical testing revealed that all the capsule prototypes could withstand forces significantly greater than expected physiological forces. Thus, the capsule with the thinnest wall (0.64 mm) is recognized as an ideal design because it allows for optimized sensitivity. Further, sensor/gel sensitivity testing showed that sensor sensitivity was not impacted by a gel thickness of up to 3 mm. Consequently, gel thicknesses of 3 mm and less are preferred for purposes of various aspects of the present invention.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A device for measuring pressure therein an area of a patient, comprising:
a capsule having an outer wall and a base surface, wherein the outer wall and the base surface cooperate to define an inner cavity configured to retain a fluid, and wherein at least a portion of the outer wall of the capsule comprises a flexible material; and
a pressure sensor configured to measure intra-capsular pressure that may be induced by any external pressure or force placed on the outer wall of the capsule.

2. The device of claim 1, wherein the capsule is configured to be positioned therein a non-fluid-filled area of the patient and measure pressure therein the non-fluid-filled area.

3. The device of claim 1, wherein the pressure sensor is positioned therein the inner cavity of the capsule.

4. The device of claim 3, wherein the pressure sensor is configured for wireless communication with an external data acquisition device.

5. The device of claim 1, wherein the flexible material is an elastomeric material.

6. The device of claim 5, wherein the elastomeric material is silicone elastomer.

7. The device of claim 1, wherein the capsule comprises a substantially hemi-spherical distal end portion, an opposing proximal end portion, and a substantially cylindrical body portion extending therebetween the proximal end portion and the distal end portion.

8. The device of claim 7, wherein a diameter of the substantially cylindrical body portion is between about 0.5 centimeters and 1.5 centimeters.

9. The device of claim 8, wherein the diameter of the substantially cylindrical body portion is about 1.2 centimeters.

10. The device of claim 7, wherein the capsule has a longitudinal axis that extends between the proximal end portion and the distal end portion, and wherein the capsule has a length taken along the longitudinal axis of between about 2 centimeters and about 3 centimeters.

11. The device of claim 10, wherein the length of the capsule is about 3 centimeters.

12. The device of claim 1, further comprising a tether member comprising a lumen having a distal end in communication with the pressure sensor and an opposing proximal end in communication with an atmospheric pressure source.

13. The device of claim 12, wherein the tether member further comprises means for providing wired communication from the pressure sensor to an external data acquisition device.

14. The device of claim 1, wherein the fluid comprises silicone gel.

15. A method for measuring intra-vaginal pressure of a patient, comprising:
providing a device for measuring the intra-vaginal pressure of the patient, comprising:
a capsule having an outer wall and a base surface, wherein the outer wall and the base surface cooperate to define an inner cavity configured to retain a fluid, and wherein at least a portion of the outer wall of the capsule comprises a flexible material; and
a pressure sensor configured to measure pressure placed on the outer wall of the capsule;
positioning the device therein a vagina of the patient above the pelvic floor of the patient; and
taking at least one pressure measurement based on the pressure placed on the outer wall of the capsule by the vagina of the patient.

16. The method of claim 15, wherein taking at least one pressure measurement comprises taking at least one pressure measurement while the patient performs at least one activity.

17. The method of claim 16, wherein the at least one activity comprises at least one of:
sitting, standing, walking, running, jumping, coughing, sneezing, performing a Valsalva maneuver, squatting, or lifting weights.

18. The method of claim 17, further comprising determining if the at least one pressure measurement differs from a predetermined pressure threshold for the at least one activity.

19. The method of claim 18, further comprising generating a treatment regimen based on the determined differences between the at least one pressure measurement and the predetermined pressure threshold for the at least one activity.

20. The method of claim 15, wherein the capsule comprises a substantially hemi-spherical distal end portion, an opposing proximal end portion, and a substantially cylindrical body portion extending therebetween the proximal end portion and the distal end portion, and wherein positioning the device therein the vagina of the patient comprises inserting the device therein the vagina such that the distal end portion of the capsule is proximate a cervix of the patient.

21. The method of claim 20, wherein the device further comprises a tether member operably connected to the proximal end portion of the capsule, wherein the tether member is configured for removal of the device from the vagina of the patient.

22. The method of claim 15, further comprising transmitting the at least one pressure measurement wirelessly to an external data acquisition device.

23. A method for measuring pressure therein a non-fluid-filled area of a patient, comprising:
providing a device for measuring the pressure therein the non-fluid-filled area of the patient, comprising:
a capsule having an outer wall and a base surface, wherein the outer wall and the base surface cooperate to define an inner cavity configured to retain a fluid, and wherein at least a portion of the outer wall of the capsule comprises a flexible material; and
a pressure sensor configured to measure pressure placed on the outer wall of the capsule;
positioning the device therein the non-fluid-filled area of the patient; and
taking at least one pressure measurement based on the pressure placed on the outer wall of the capsule by the non-fluid-filled area of the patient.

24. The method of claim 23, further comprising determining if the at least one pressure measurement differs from a predetermined pressure threshold.

25. The method of claim 24, further comprising generating a treatment regimen based on the determined difference between the at least one pressure measurement and the predetermined pressure threshold.

26. A device for measuring pressure therein a non-fluid-filled area of a patient, comprising:
a capsule having an outer wall and a base surface, wherein the outer wall and the base surface cooperate to define an inner cavity configured to retain a fluid, and wherein at least a portion of the outer wall of the capsule comprises a flexible material; and
a pressure sensor configured to measure external pressure placed on the outer wall of the capsule.
